(19) United States
(12) Patent Application Publication
(10) Pub. No.: US 2007/0281366 A1
(43) Pub. Date: Dec. 6, 2007

(54) Target molecule recognition polymer and method for producing the same

(75) Inventors: Yuichiro Shimizu, Kidugawa-shi (JP); Takateru Matsunaga, Ichikawa-shi (JP)

Correspondence Address: BIRCH STEWART KOLASCH & BIRCH, PO BOX 747, FALLS CHURCH, VA, 22040-0747, US

(73) Assignee: Sharp Kabushiki Kaisha

(21) Appl. No.: 11/724,196

(22) Filed: Mar. 15, 2007

(30) Foreign Application Priority Data
Apr. 3, 2006 (JP) 2006-102176

(51) Int. Cl. G01N 33/53

(52) U.S. Cl. 436/501

(57) ABSTRACT

A mixture of one or more target molecules and functional monomers having functional groups, which are able to interact with the target molecules, is polymerized so as to form a target molecule recognition polymer (MIP) complex to which target molecules are bound, and a functional group which is contained in the MIP complex but is not bound to the target molecule is deactivated. This makes it possible to suppress binding of a non-target molecule that can be bound to the MIP by weak interaction. Thus, it is possible to provide an MIP exhibiting a high selectivity even when high molecular weight molecules like biomolecules are used as the target molecules.

This Nonprovisional application claims priority under 35 U.S.C. § 119(a) on Patent Application No. 102176/2006 filed in Japan on Apr. 3, 2006, the entire contents of which are hereby incorporated by reference.

### FIELD OF THE INVENTION

The present invention relates to a target molecule recognition polymer and a method for producing the same. More specifically, the present invention relates to (a) a target molecule recognition polymer which is able to bind to a target molecule in a selective manner and obtained by a molecular imprinting process, and (b) a method for producing the same.

### BACKGROUND OF THE INVENTION

As a method for producing a target molecule recognition polymer which is able to specifically bind to a target molecule, known is the technique of “molecular imprinting” in which a molecule itself is allowed to design functional sites of a target molecule recognition polymer. The principle of the molecular imprinting is very simple. The principle of the molecular imprinting is that in synthesizing a crosslinking polymer having specific binding sites with respect to a target molecule, a mixture of (i) a monomer for polymer-synthesis and (ii) a target molecule is polymerized to obtain a polymer, and the target molecule is washed away from the polymer that has been obtained after the polymerization, thus forming binding sites in the polymer which binding sites are complementary to the target molecule. A typical molecular imprinting is such that if there are characteristic functional groups in a target molecule, the target molecule and a monomer (functional monomer) which is able to interact with the functional groups by noncovalent binding are polymerized with a crosslinking agent which serves as a matrix of a polymer, or that a prepared complex of a target molecule with a functional monomer is polymerized with a crosslinking agent. After the polymerization, an obtained polymer is swollen and cleaned by a solution (refining solution) that has the effect of having the target molecule removed from the polymer. As a result, functional groups which are able to interact with the target molecule remain in a state like a template of the target molecule in the polymer at the area where the target molecule has been removed from the polymer. In this manner, the polymer which is able to specifically bind to the target molecule is obtained. That is, functional monomer-derived functional groups are arranged in a matrix of a crosslinking agent-derived polymer in such a manner so as to fit into the target molecule and recognize the characteristic functional groups in the target molecule. Areas around the functional groups localized in the polymer become sites that specifically recognize and bind to the target molecules.

As disclosed in Patent document 1 (Japanese Unexamined Patent Publication No. 506320/1996 (Tokuhyohei 8-506320), published on Jul. 9, 1996) and Non-patent document 1 (Kazuko Hirayama, Martin Burow, Yoshitomi Morikawa, and Norihiko Minoura (1998) Synthesis of Polymer-Coated Silica Particles with Specific Recognition Sites for Glucose Oxidase by the Molecular Imprinting Technique. Chemistry Letters, 731-732), the imprinting technique using biomolecules as target molecules has been recently studied. Target molecule recognition polymers (hereinafter also referred to as imprint polymers) have the advantages of having a unique stability that is more excellent than natural biomolecules and allowing for handling under conditions unsuitable for the use of biomolecules (e.g. high temperature, organic solvent, and extreme pH). Moreover, a target molecule recognition polymer is prepared by a relatively simple method and at low cost.

However, imprint polymers disclosed in Patent document 1 and Non-patent document 1 have the problem of a poor selectivity.

For example, like imprint polymers of Non-patent document 1, an imprint polymer produced by molecular imprinting using glucose oxidase that is a protein as target molecules has the problem of having nonspecific bonds much more than specific bonds, i.e. having an extremely poor selectivity. In Non-patent document 1, an experiment is conducted to recombine (a) an imprint polymer obtained by using glucose oxidase as target molecules and (b) glucose-6-phosphatedehydrogenase that is similar to glucose oxidase. The result of the experiment is that almost all glucose-6-phosphatedehydrogenase added are bound to an imprint polymer by specific adsorption. The reason for the result is considered that since high molecular weight molecules like biomolecules have functional groups much more than low molecular weight molecules and have much more functional groups at sites other than binding sites of the imprint polymer, an extremely large number of non-target molecules are bound to the imprint polymer by physical adsorption and weak interaction.

### SUMMARY OF THE INVENTION

The present invention has been attained in view of the above problems, and an object of the present invention is to provide a target molecule recognition polymer (imprint polymer) which can bind to a target molecule and exhibit a high selectivity even when high molecular weight molecules like biomolecules are used as target molecules, and a method for producing the target molecule recognition polymer.

In order to solve the above problems, a method for producing a target molecule recognition polymer according to the present invention (hereinafter referred to as first producing method) includes: a polymerization step of polymerizing a mixture of one or more target molecules and functional monomers having functional groups, which are able to interact with the target molecules, so as to form a target molecule recognition polymer complex to which target molecules are bound; and a refining step of refining the target molecule recognition polymer complex by having one or more target molecules removed from the target molecule recognition polymer complex, so as to obtain a target molecule recognition polymer, the method further comprising: a deactivation step of deactivating a functional group which is contained in the target molecule recognition polymer complex but is not bound to the target molecule.

With the above arrangement, it is possible to provide a target molecule recognition polymer which realizes a high selectivity even when high molecular weight molecules like biomolecules are used as the target molecules.

More specifically, according to the first producing method of the present invention, a functional group which is able to bind to the target molecule contained in the target molecule recognition polymer (imprint polymer) but does not exist at a binding site where a functional group is bound to the target molecule is deactivated. Thus, it is possible to suppress binding of a non-target molecule that can possibly bind to the target molecule recognition polymer by weak electrostatic interaction.

Note that the “interaction” herein collectively means interactions such as electrostatic interaction between negative charge and positive charge, hydrogen bonding, hydrophobic interaction. Further, “to deactivate” means to disable binding of a functional group which can interact with or bind to the target molecule and other molecules so that the functional group cannot be bound to the target molecule.

Especially, there was the problem that in a case where high molecular weight molecules having numerous functional groups, like biomolecules, are used as the target molecules, the high molecular weight molecule is bound, by weak electrostatic interaction, to a functional group contained in the polymer which functional group is not originally involved in the binding to the target molecule. However, according to the first producing method of the present invention, even when such high molecular weight molecules are the target molecules, the high molecular weight molecules are accurately recognized, thereby suppressing interactions with other substances (non-target molecules). This is because the functional group which is not at the binding site of the target molecule recognition polymer for the high molecular weight molecule is deactivated. That is, according to the first producing method of the invention, it is possible to provide a target molecule recognition polymer which realizes a high selectivity.

Further, according to the first producing method of the invention, such a deactivation process is carried out with respect to the target molecule recognition polymer complex. With this arrangement, it is possible to accurately deactivate the functional group which is not at the binding site of the target molecule recognition polymer.

As described above, according to the first producing method of the invention, it is possible to provide a target molecule recognition polymer which realizes a high selectivity. Thus, it is possible to provide a target molecule recognition polymer which realizes a high selectivity even when high molecular weight molecules like biomolecules are used as the target molecules.

Further, in order to solve the foregoing problems, another method for producing target molecule recognition polymers according to the present invention (hereinafter referred to as second producing method) includes: a polymerization step of polymerizing a mixture of one or more target molecules and functional monomers having functional groups, which are able to interact with the target molecules, so as to form a target molecule recognition polymer complex to which target molecules are bound; a refining step of refining the target molecule recognition polymer complex by having one or more target molecules removed from the target molecule recognition polymer complex, so as to obtain a target molecule recognition polymer; a complex forming step of mixing the target molecule recognition polymer that has been obtained in the refining step with target molecules in predetermined quantity, so as to form a complex of the target molecule recognition polymer with a target molecule; and a deactivation step of deactivating a functional group which is contained in the target molecule recognition polymer complex that has been obtained in the complex forming step, but is not bound to the target molecule.

With the above arrangement, it is possible to provide a target molecule recognition polymer which realizes a high selectivity even when high molecular weight molecules like biomolecules are used as the target molecules.

More specifically, according to the second producing method of the present invention, after target molecules have been removed from the target molecule recognition polymer complex, a complex of the target molecule recognition polymer with a target molecule are formed, and the functional group contained in the complex which functional group has not been bound to the target molecule is deactivated. This makes it possible to suppress binding of the non-target molecule that can possibly bind to the target molecule recognition polymer by weak electrostatic interaction.

Further, according to the second producing method of the present invention, the target molecules in a predetermined concentration are mixed in forming the complex of the target molecule recognition polymer and the target molecule. Here, the “target molecules in predetermined quantity” are target molecules not more than one-tenth of the quantity, preferably not more than one-thousands of the target molecules to be mixed with the functional monomers in the polymerization step. The quantity of target molecules is smaller than a total number of sites that exist in the target molecule recognition polymer obtained by the refining step and can interact with target molecules. With this arrangement, the target molecules are bound to sites which have a plurality of functional groups and are better suited to bind to the target molecules in the polymer. In such a complex, when a functional group which is not bound to the target molecule is deactivated, it is possible to obtain a target molecule recognition polymer having selectivity higher than the target molecule recognition polymer obtained by the first producing method of the present invention.

As described above, according to the second producing method of the invention, it is possible to provide a target molecule recognition polymer which realizes a high selectivity. Thus, it is possible to provide a target molecule recognition polymer which realizes a high selectivity even when high molecular weight molecules like biomolecules are used as the target molecules.

In addition to the above arrangement, the method for producing a target molecule recognition polymer of the present invention preferably includes: a cleaning step of removing (a) a target molecule which is physically adsorbed with the target molecule recognition polymer complex and/or (b) a target molecule and a functional monomer both of which have not made up the target molecule recognition polymer complex.

Thus, since the target molecule which is physically adsorbed to the target molecule recognition polymer complex is removed, it is possible to efficiently deactivate a functional group which is not involved in binding to the target molecule in the deactivation step. By removing redundant target molecules and functional monomers which have not made up the target molecule recognition polymer complex, it is also possible to enhance the efficiency of deactivation.

Therefore, according to the above arrangement, it is possible to enhance selectivity of the target molecule recognition polymer obtained by the method of the present invention.

Further, in addition to the above arrangement, the method for producing a target molecule recognition polymer according to the present invention is preferably such that in the refining step used is a dissociation solution which dissociates the target molecules from the target molecule recognition polymer complex at a predetermined dissociation strength, and the method further comprises: a pretreatment step of, by using a solution having a dissociation strength lower than the predetermined dissociation strength, removing (a) a target molecule which is physically adsorbed with the target molecule recognition polymer complex and/or (b) part of target molecules which is bound to the complex by interaction with the functional groups.

As described above, the present invention includes the pretreatment step using a solution having dissociation strength lower than a predetermined dissociation strength of a dissociation solution used to dissociate the target molecules from target molecule recognition polymer complex in the refining step. This makes it possible to remove part of target molecules which is bound to the target molecule recognition polymer complex as well as a target molecule which is physically adsorbed with the complex, by using the solution used in the pretreatment step. Here, the “part of target molecules” means a target molecule which is not well suited to bind to the complex, among target molecules which are bound to the complex. In other words, the “part of target molecules” means a target molecule which is not suited to bind to the complex so that the target molecule is dissociated from the target molecule recognition polymer complex by a solution having dissociation strength lower than that of a dissociation solution used in the refining step.

Thus, a target molecule which is not well suited to bind to the complex is removed by using a solution having a dissociation strength weaker than that of a dissociation solution used in the refining step, so that it is possible to deactivate a functional group to which the target molecule has been bound. In other words, only a functional group which is better suited to bind to a target molecule remains without being deactivated. This makes it possible to enhance selectivity of the target molecule recognition polymer obtained by the method of the present invention.

Further, the method for producing a target molecule recognition polymer according to the present invention is such that primary amine having a molecular weight of not more than several thousands is used in the deactivation step.

With the above arrangement, it is possible to efficiently carry out a functional group deactivating process.

Further, primary amine having a molecular weight of not more than several thousands is suitable for a case where monomers negatively charged, like acrylic acid, are used as the functional monomers.

Still further, the method for producing a target molecule recognition polymer according to the present invention is preferably such that the primary amine is ethanolamine or tris(hydroxymethyl)aminomethane.

With the above arrangement, it is possible to efficiently carry out a functional group deactivating process.

Further, ethanolamine or tris(hydroxymethyl)aminomethane is suitable for a case where monomers negatively charged, like acrylic acid, are used as the functional monomers.

Still further, the method for producing a target molecule recognition polymer according to the present invention is such that acetic anhydride is used in the deactivation step.

With the above arrangement, it is possible to efficiently carry out a functional group deactivating process.

Further, acetic anhydride is suitable for a case where basic monomers are used as the functional monomers.

Still further, the method for producing a target molecule recognition polymer according to the present invention is such that a water-soluble polymer is used in the deactivation step, and more specifically, the water-soluble polymer is polyvinyl alcohol or polyethyleneglycol.

With the above arrangement, it is possible to efficiently carry out a functional group deactivating process.

Further, polyvinyl alcohol or polyethyleneglycol is suitable for a case where hydrophobic monomers are used as the functional monomers.

Still further, the method for producing a target molecule recognition polymer according to the present invention is preferably such that the target molecules are high molecular weight molecules each having a molecular weight of not less than ten thousands. More specifically, it is preferable that the target molecules are biomolecules.

Examples of the biomolecules are protein and DNA. Even when such high molecular weight molecules are the target molecules, the producing method of the present invention makes it possible to produce a target molecule recognition polymer having a high selectivity, thus enabling recognition of a target molecule with high accuracy.

Further, the method for producing a target molecule recognition polymer according to the present invention is preferably such that a crosslinking agent is further mixed in the polymerization step.

The above arrangement enables effective crosslinking of the functional monomers, thus providing an excellent target molecule recognition polymer.

Further, in order to solve the foregoing problems, a target molecule recognition polymer according to the present invention is produced by the above producing method.

With the above arrangement, it is possible to provide a target molecule recognition polymer which realizes a high selectivity even when high molecular weight molecules like biomolecules are used as the target molecules.

In order to solve the foregoing problems, the target molecule recognition polymer according to the present invention is a target molecule recognition polymer obtained by polymerizing functional monomers having functional groups, which are able to interact with target molecules, wherein part of the functional groups is deactivated.

With the above arrangement, it is possible to provide a target molecule recognition polymer which realizes a high selectivity even when high molecular weight molecules like biomolecules are used as the target molecules.

The present invention also includes a sensor for detecting target molecules, the sensor comprising a target molecule recognition polymer produced by the above producing method or a target molecule recognition polymer having the above features.

Additional objects, features, and strengths of the present invention will be made clear by the description below. Further, the advantages of the present invention will be evident from the following explanation in reference to the drawings.

### BRIEF DESCRIPTION OF THE DRAWINGS

FIG. 1 is a block diagram illustrating a method for producing a target molecule recognition polymer, according to First Embodiment of the present invention.

FIG. 2 is a view illustrating structures of a monomer and a polymer obtained by the producing method shown in FIG. 1.

FIG. 3 is a block diagram illustrating a method for producing a target molecule recognition polymer, according to Second Embodiment of the present invention.

FIG. 4 is a block diagram illustrating a method for producing a target molecule recognition polymer, according to Third Embodiment of the present invention.

FIG. 5 is a block diagram illustrating a method for producing a target molecule recognition polymer, according to Fourth Embodiment of the present invention.

### DESCRIPTION OF THE EMBODIMENTS

The following will describe one embodiment of a target molecule recognition polymer according to the present invention and a method for producing the target molecule recognition polymer.

Note that although the following description includes various technically preferable limitations to carry out the present invention, the scope of the present invention is not limited solely by the following embodiment and drawings.

<Target Molecule Recognition Polymer>

A target molecule recognition polymer (also termed as Molecular Imprint Polymer, and hereinafter referred to as MIP) according to the present invention is obtained by polymerization of functional monomers having functional groups, which interact with target molecules. An MIP of the present invention is an MIP which is able to specifically bind to a target molecule, the MIP produced by polymerizing a mixture of one or more target molecules and functional monomers so as to form an MIP complex to which target molecules are bound, and removing the target molecules from the MIP complex so that functional groups which are able to interact with target molecules remain in a state like a template of the target molecule in the polymer at an area where the target molecules have been removed from the MIP complex. Details of the MIP of the present invention are described later.

Such an MIP can be turn to various MIPs by changing type of target molecules as used. For example, by (a) using, as the target molecules, drug, metabolic product, nucleotide, nucleic acid, carbohydrate, protein, hormone, toxic substance, or steroid and (b) using functional monomers having functional groups capable of interaction with such target molecules, it is possible to provide an MIP which is able to specifically bind to the respective target molecules.

Concrete examples of the MIPs include artificial antibodies. Antibodies are normally produced by immunizing animals with the corresponding antigen leading to polyclonal antibodies, or by using fused cells (B cells) allowing the obtained cell lines to produce monoclonal antibodies. However, it is difficult to sufficiently enhance the efficiency of production of antibodies since production of antibodies is a time-consuming method and needs to undergo various complicated steps. Furthermore, the antibodies must be handled and stored with some care since the antibodies are unstable biomolecules. On the contrary, artificial antibodies realized by MIPs function in the same manner as antibodies and are produced by relatively simple steps as described above since the artificial antibodies carry specific binding sites, as the above-mentioned functional groups, mimicking the properties of antibodies. Additionally, the artificial antibodies have the advantage of being stable.

Further, the MIP according to the present invention is characterized in that part of functional groups which are able to bind to a target molecule is deactivated. The “part of the functional groups” herein means a functional group which is able to bind to a target molecule and does not exists at a binding site where functional groups are bound to a target molecule in an MIP complex to which a target molecule is being bound. Further, the “deactivation” means to disable binding of a functional group which can bind to a target molecule so that the functional group cannot be bound to the target molecule.

Thus, by deactivating a functional group which does not contribute to binding to a target molecule in an MIP complex, it is possible to suppress binding of non-target molecule which is able to bind to a target molecule recognition polymer by weak interaction, i.e. a molecule which is not the target molecule. This makes it possible to enhance selectivity of an MIP.

<Method for Producing a Target Molecule Recognition Polymer>

The following will describe one embodiment of a MIP producing method according to the present invention.

### First Embodiment

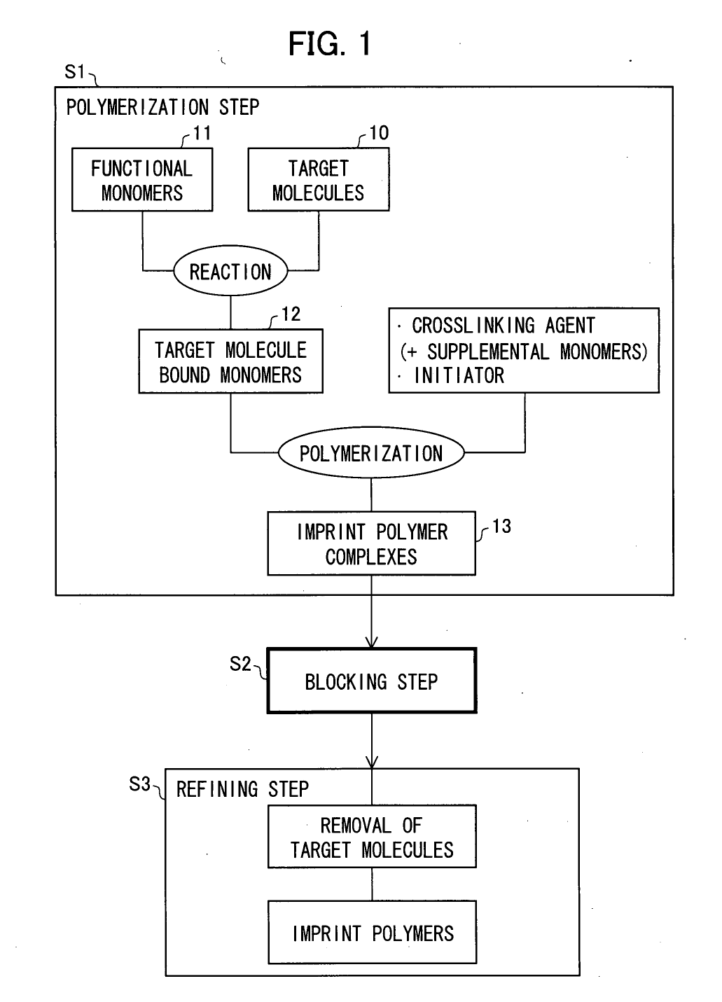

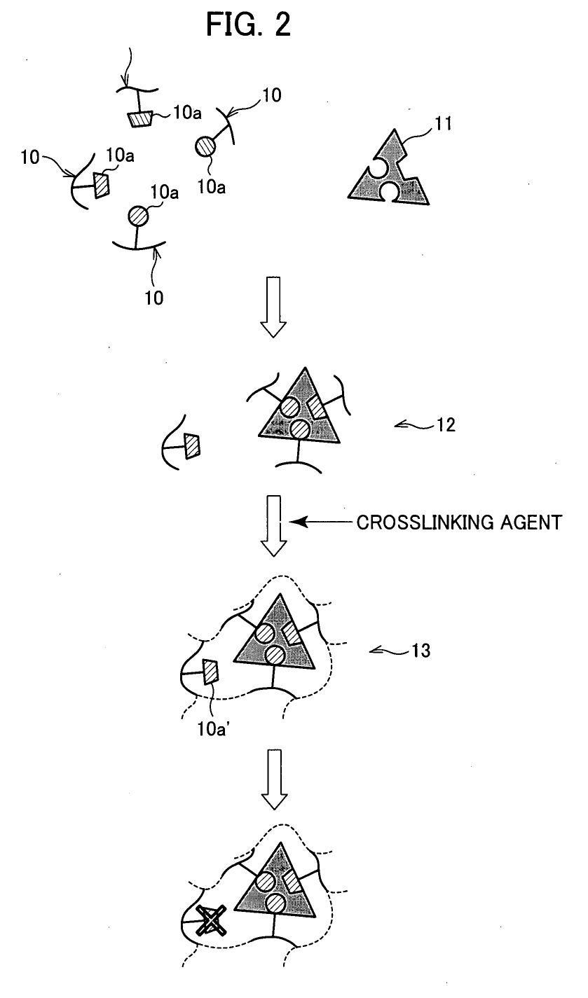

A production method of the present embodiment will be explained with reference to FIGS. 1 and 2.

FIG. 1 is a block diagram illustrating a MIP producing method which is one embodiment of the present invention. FIG. 2 is a view illustrating the state of a monomer or a polymer in a polymerization step in a production process illustrated in FIG. 1.

As illustrated in FIG. 1, a production method of the present embodiment includes: a polymerization step S1 of polymerizing a mixture of one or more target molecules and functional monomers having functional groups, which are able to interact with the target molecule, so as to form an MIP complex which the target molecules are interacted with and bound to; a blocking step (deactivation step) S2 of blocking (deactivating) a functional group which is not interacted with the target molecule in the MIP complex; and a refining step S3 of refining the MIP complex by removing the target molecules which are bound to the MIP complex from the MIP complex, so as to obtain an MIP.

In the polymerization step S1, as illustrated in FIG. 1, target molecules are mixed with functional monomers having functional groups, which are able to interact with the target molecules. This forms a target molecule bound monomer 12 in which a target molecule 11 is bound to functional groups 10a of their respective functional monomers 10 by interactions, as illustrated in FIG. 2.

Then, in the polymerization step S1, the target molecule bound monomer and a crosslinking agent are mixed and polymerized, as illustrated in FIG. 1. This allows functional monomers of the target molecule bound monomer 12 to be crosslinked to each other, thereby forming an MIP complex 13, as illustrated in FIG. 2.

The “interaction” herein collectively means interactions such as electrostatic interaction between positive charge and negative charge, hydrogen bonding, hydrophobic interaction. The electrostatic interaction between positive charge and negative charge is such that positively charged groups such as amino group and imidazole group, which are included in a protein, are interacted with negatively charged groups such as carboxyl group and phosphate group. On the other hand, negatively charged groups such as carboxyl group, which is included in a protein, are interacted with positively charged groups such as amino group, imino group, and tertiary amino group. That is, to utilize the electrostatic interaction between positive charge and negative charge, functional monomers having vinyl groups, such as acrylic acid, methacrylic acid, or N,N-dimethylaminopropylacrylamide, are selected as the functional monomers. The selected functional monomers are preferably water-soluble.

Examples of the functional monomer 10 include hydrophilic functional polymers such as acrylic acid, methacrylic acid, itaconic acid, trifluoromethacrylic acid, vinylpyridine, vinylimidazole, vinylbenzoic acid, 4-vinylbenzylimide diacetate, 2-acrylamide-2-methyl-1-propanesulfonic acid, and 2,6-bisacrylamide pyridine. Note that these functional monomers may be used in combination.

The target molecule 11 can be a drug, a metabolic product, nucleotide, nucleic acid, carbohydrate, a protein, a hormone, a toxic substance, or steroid.

The crosslinking agent can be a conventionally known crosslinking agent. Specifically, the crosslinking agent can be N,N′-methylenebisacrylamide.

Examples of the polymerization reaction include radical polymerization (mass polymerization, suspension polymerization, solution polymerization, emulsion polymerization, seed polymerization, dispersion polymerization, reverse suspension polymerization, soap-free polymerization), ion polymerization (anionic polymerization, cationic polymerization), coordination polymerization, ring-opening polymerization, and condensation polymerization.

As illustrated in FIG. 1, polymerization is carried out by using the crosslinking agent in the present embodiment. However, this is not the only possibility of the present invention. Alternatively, the MIP complex 13 may be formed by polymerization of functional monomers of the target molecule bound monomer 12 without using the crosslinking agent.

In the present embodiment, after the target molecule bound monomer 12 has been formed, the target molecule bound monomer 12 is mixed with the crosslinking agent. However, this is not the only possibility of the present invention. Alternatively, the functional monomers 10, the target molecule 11, and the crosslinking agent may be mixed and polymerized together.

Further, in the present embodiment, only the crosslinking agent is mixed with the target molecule bound monomer 12 at the polymerization. However, this is not the only possibility of the present invention. Alternatively, in addition to the crosslinking agent, a supplemental monomer may be mixed therewith, or an initiator may be mixed therewith. The supplemental monomer can be a conventionally known one that assists in the formation of a crosslinked structure. Specifically, the supplemental monomer can be acrylamide. Further, the initiator can be a conventionally known one, provided that it acts for the initiation of polymerization. Specifically, the initiator can be sodium persulfate.

Conventionally, an MIP is completed by, in the refining step S3, removing the target molecules from the MIP complex obtained in the polymerization step S1. However, in the MIP produced by the conventional method, there is the possibility that a non-target molecule can interact with and bind to a functional group 10a′ of the MIP. Thus, the conventional MIP has the problem of a poor selectivity since the MIP is specifically bound to a molecule other than the target molecule.

On the contrary, the present embodiment includes the blocking step S2, as illustrated in FIG. 1. With this arrangement, an obtained MIP does not bind to a molecule that is not the target molecule. Details of the blocking step S2 will be described below.

In the blocking step S2, the functional groups 10a which are provided in the MIP complex 13 that has been obtained in the polymerization step S1 are deactivated. In the present embodiment, it should be noted that in the blocking step S2, an active functional group 10a′ which does not contribute to interaction with the target molecule 11 (hereinafter referred to as free functional group) is deactivated among the functional groups 10a provided in the MIP complex 13 that has been obtained in the polymerization step S1, as illustrated in FIG. 1. FIG. 2 illustrates that the free functional group 10a′ in the MIP complex 13 has been blocked in the blocking step S2.

When the MIP complex 13 undergoes the blocking step S2, the active functional group which has remained in the MIP complex 13 is deactivated. Therefore, an MIP of the present embodiment completed after the subsequent steps as will be described later is not able to interact with a non-target molecule, unlike the MIP produced by the conventional method. In other words, the MIP of the present embodiment is not specifically bound to a non-target molecule.

Examples of the blocking method include non-covalent binding (e.g. hydrophobic bonding or electrostatic interactions) and covalent binding. A choice between non-covalent binding and covalent binding should be appropriately made in view of functional monomers as used. However, in consideration of a cleaning step, covalent binding is preferable to non-covalent binding.

Specifically, in a case where the functional monomers are negatively charged monomers such as carboxyl group and aldehyde group, a blocking agent can be low molecular primary amine having a molecular weight of not more than several thousands. Examples of the low molecular primary amine include ethanolamine, glycine, and tris(hydroxymethyl)aminomethane. Since primary amine binds to an active group like a carboxyl group to deactivate the active group, it is possible to block the free functional group 10a′ illustrated in FIG. 2. In a case where the functional monomer is a basic monomer including a group like an amino group as the free functional group 10a′, the blocking agent can be acetic anhydride. By using acetic anhydride, it is possible to deactivate the amino group by acetylating the amino group. Further, in a case where the functional monomer is a hydrophobic monomer having a highly hydrophobic functional group, the blocking agent can be a substance having low molecular weight molecules, such as polyvinyl alcohol or polyethyleneglycol. In this case, it is possible to block the free functional group 10a′ by having the low molecular weight molecules adsorbed with the free functional group 10a′. In this method, since the functional group is just blocked by non-covalent binding rather than covalent binding, it is necessary to ascertain whether the low molecular weight molecules are not separated in pretreatment of the MIP.

Note that in the blocking step S2 of the present embodiment, as illustrated in FIG. 2, a structure of the free functional group 10a′ is changed so that activity of the free functional group 10a′ is negated. However, this is not the only possibility of the present invention. Alternatively, for example, the free functional group 10a′ itself may be removed for deactivation. This makes it possible to negate activity of the free functional group 10a′ as in the blocking step of the present embodiment.

After the MIP complex 13 is subjected to blocking by the above-mentioned method, the blocking agent is removed by means of a suitable solvent. For example, the solvent can be 50 mM Tris-HCl (pH 8.0) as shown in Example described later. Then, a refining step in which the target molecules bound to the MIP complex are removed is carried out (S3 in FIG. 1). This refines the MIP according to the present embodiment.

In the refining step S3, the target molecules are removed by suitably using a solution capable of removing the target molecules bound to the MIP complex to complete the MIP according to the present invention. In the refining step S3, it is possible to remove the target molecules from the MIP complex by using, for example, a solution having a high salt concentration or a solution having a low pH.

Thus, according to the producing method of the present embodiment, it is possible to deactivate a problem functional group which is not the functional group bound to the target molecule at the binding site in the MIP. Thus, it is possible to suppress binding of the non-target molecule that can possibly bind to the target molecule recognition polymer by weak electrostatic interaction. With this arrangement, even when high molecular weight molecules are used as the target molecules, it is possible to accurately recognize the high molecular weight molecules since the functional group other than the functional group bound to the target molecule at the binding site in the MIP is deactivated. Thus, it is possible to suppress interaction with other substance (non-target molecule). In other words, with the producing method of the present embodiment, it is possible to provide a MIP which realized a high selectivity.

Further, according to the producing method of the present embodiment, it is possible to accurately deactivate the functional group at other-than-binding site in the MIP by subjecting the MIP complex to such a deactivation process.

### Second Embodiment

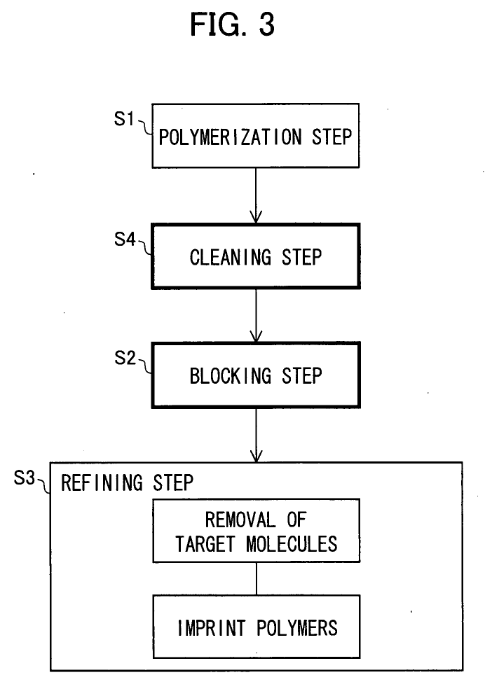

The following will describe another embodiment of the present invention with reference to FIG. 3. Note that, for the purpose of explanation of differences from the First Embodiment, substances that are identical with those described in the First Embodiment are given the same reference numerals and explanations thereof are omitted here.

FIG. 3 is a block diagram illustrating a MIP producing method of the present embodiment. In the First Embodiment, the polymerization step S1 (FIG. 1) is followed by the blocking step S2. On the contrary, according to a producing method of the present embodiment, as illustrated in FIG. 3, a polymerization step S1 is followed by a cleaning step S4, and the cleaning step S4 is followed by a blocking step S2.

In the cleaning step S4, a polymerization reaction solution, which is used to form an MIP complex 13 in the polymerization step S1, is substituted for a suitable solution (hereinafter referred to as cleaning solution). This makes it possible to remove a functional monomer 10 and a target molecule 11 which have remained in the polymerization reaction solution without having made up the MIP complex 13, and to separate the functional monomer 10 and the target molecule 11 from the MIP complex 13. Further, by the cleaning step S4, it is possible to wash away a target molecule 11 which is not specifically interacted with the MIP complex 13 but is physically adsorbed with the MIP complex 13.

The cleaning solution used in the cleaning step S4 is not particularly limited, provided that it does not remove a target molecule 11 which is constitutive part of the MIP complex 13. Further, the cleaning solution can be appropriately selected in accordance with functional monomer 10 and target molecules 11 as used in the production. The cleaning solution is normally a buffer for use in synthesis (specifically, phosphate buffer or tris-buffer).

A concrete operation in the cleaning step S4 is not particularly limited. For example, the polymerization reaction solution may be gradually substituted for the cleaning solution. Alternatively, the MIP complex 13 may be cleaned with the cleaning solution after all of the polymerization reaction solution has been removed by a method like suction filtration.

Note that a complete substitution of the polymerization reaction solution for the cleaning solution makes it possible to obtain the effect of the cleaning step of the present embodiment. The amount of polymerization reaction solution to be substituted should be appropriately determined in order to remove (a) the functional monomer 10 and target molecule 11 both of which have remained in the polymerization reaction solution and (b) the target molecule 11 which has been physically absorbed the MIP complex 13.

In the MIP producing method of the present embodiment, the above-mentioned cleaning step S4 is followed by the blocking step S2 and the refining step S3. Explanation of the blocking step S2 and the refining step S3 are omitted here because both of them have been explained in the First Embodiment.

Thus, the present embodiment is characterized in that the blocking step S2 follows the cleaning step S4 in which the MIP complex 13 is cleaned. With this arrangement, in the present embodiment, it is possible to carry out the blocking step S2 in a state where there occurs less non-specific adsorption than in the First Embodiment. Thus, it is possible to efficiently deactivate active functional group 10a′ (free functional group) that does not contribute to the interaction with the target molecule 11, among the functional groups 10a provided in the MIP complex 13. This enhances the efficiency of blocking, thus enhancing selectivity of a produced MIP.

### Third Embodiment

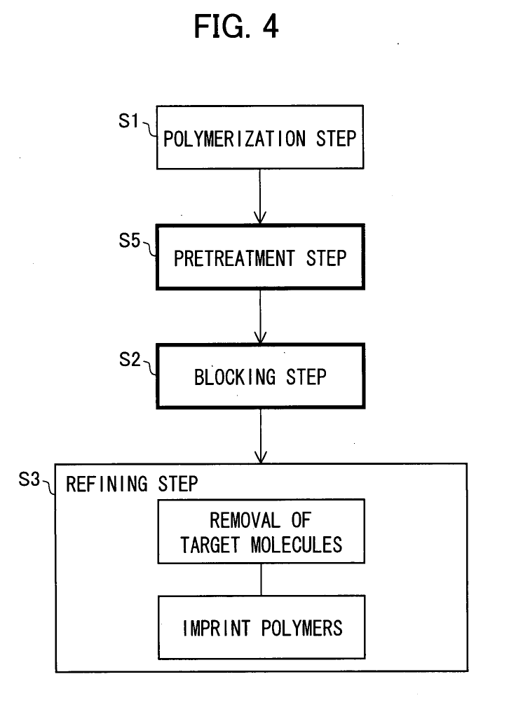

The following will describe still another embodiment of the present invention with reference to FIG. 4. Note that, for the purpose of explanation of differences from the First Embodiment, substances that are identical with those described in the First Embodiment are given the same reference numerals and explanations thereof are omitted here.

FIG. 4 is a block diagram illustrating a MIP producing method of the present embodiment. In the First Embodiment, the polymerization step S1 is followed by the blocking step S2, as illustrated in FIG. 1. On the contrary, according to a producing method of the present embodiment, a polymerization step S1 is followed by a pretreatment step S5, and the pretreatment step S5 is followed by a blocking step S2, as illustrated in FIG. 4.

As illustrated in FIG. 2, a target molecule 11 has a plurality of functional groups, which interact with functional groups 10a of their respective functional monomers 10. However, in some cases, polymerization is carried out in such a manner that part of functional groups in the target molecule 11 of the MIP complex 13 (FIG. 2) does not interact with the functional groups 10a. That is, in the MIP complex 13 that has been obtained in the polymerization step S1, there exist a target molecule bound to the MIP complex 13 by relatively weak interaction (not shown in FIG. 2). In the preset embodiment, the target molecule bound to the MIP complex 13 by (relatively) weak interaction is removed from the MIP complex 13 by the pretreatment step S5 which is carried out before the blocking step S2.

In the pretreatment step S5, a solvent which is capable of removing the target molecule bound to the MIP complex 13 by weak interaction is used. Specifically, the solvent is a solution (hereinafter referred to as pretreatment solution) having a removal strength (dissociation strength) lower than a target molecule removing (dissociating) strength of a solution (refining solution in Example) for use in removing the target molecule 11 from the MIP complex 13 having been subjected to blocking in a refining step that is the last step in the producing method of the present embodiment. For example, the pretreatment solution used in Example described later is a solution having a higher salt concentration than a solution used in the refining step. This makes it possible to obtain the MIP complex 13 from which the target molecule having been bound to the MIP complex 13 by weak interaction is removed.

A concrete operation in the pretreatment step S5 is not particularly limited, provided that the MIP complex 13 is treated with the pretreatment solution in the pretreatment step S5. For example, treatment with the pretreatment solution is carried out by using a column or by suction filtration.

Further, in the pretreatment step S5, it is possible not only to remove the target molecule bound to the MIP complex by weak interaction from the MIP complex, but also to remove (a) the functional monomer and target molecule both of which have remained in the polymerization reaction solution without having made up the MIP complex and (b) the target molecule which has been physically absorbed with the MIP complex 13, although (a) and (b) are removed in the cleaning step S4 of the Second Embodiment.

As described above, the producing method of the present embodiment includes the pretreatment step S5 which uses a pretreatment solution having a lower dissociation strength than a predetermined dissociation strength of a dissociation solution which is used to dissociate the target molecule from the MIP complex in the refining step S3. By using the pretreatment solution, it is possible to remove the target molecule which is not well suited to bind to the MIP complex as well as the target molecule physically adsorbed with the MIP complex. With this arrangement, in the blocking step S2 following the pretreatment step S5, it is possible to block the (functional monomer derived) functional group in the MIP complex which functional group has been bound to the target molecule before being removed. In other words, only the functional group which is better suited to bind to the target molecule remains without being blocked. Thus, it is possible to enhance selectivity of an MIP obtained by the refining step S3.

### Fourth Embodiment

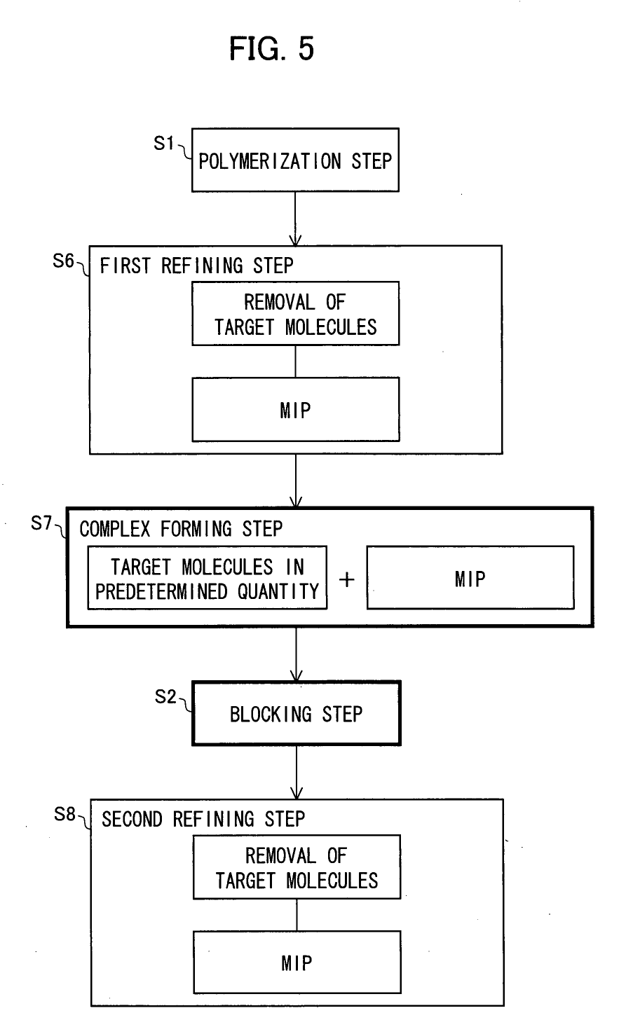

The following will describe still another embodiment of the present invention with reference to FIG. 5. Note that, for the purpose of explanation of differences from the First Embodiment, substances that are identical with those described in the First Embodiment are given the same reference numerals and explanations thereof are omitted here.

FIG. 5 is a block diagram illustrating a MIP producing method of the present embodiment. In the First Embodiment, the polymerization step S1, the blocking step S2, and the refining step S3 are carried out as illustrated in FIG. 1. On the contrary, according to a producing method of the present embodiment, as illustrated in FIG. 5, after an MIP complex is formed in a polymerization step S1, target molecules are removed to obtain a MIP (temporary MIP) in a first refining step (refining step) S6. Subsequently, a complex forming step S7, a blocking step S2, and a second refining step S8 are carried out.

In the present embodiment, after an MIP complex 13 (FIG. 1) is obtained in the polymerization step S1 described in the First Embodiment, target molecules are removed from the MIP complex to obtain an MIP in the first refining step S6, without the blocking step. Note that the first refining step S6 is identical with the refining step S3 described in the First Embodiment, and explanations thereof is omitted here.

Next, the complex forming step S7 is carried out. In the complex forming step S7, the MIP obtained in the first refining step S6 is mixed with target molecules to form an MIP complex.

Here, it should be noted that the quantity of target molecules to be interacted with the MIP in the complex forming step S7 is smaller than that of target molecules 11 to be mixed with the functional monomers 10 in the polymerization step S1. Specifically, the quantity of target molecules to be mixed with the MIP in the complex forming step S7 is not more than one-tenth of the quantity, preferably not more than one-thousands of the target molecules 11 to be mixed in the polymerization step S1. The quantity of target molecules to be mixed with the MIP in the complex forming step S7 is smaller than a total number of sites that exist in the MIP obtained by the first refining step and are able to interact with target molecules.

Thus, with adjustment of a mixture quantity of target molecules in the complex forming step S7, mixed target molecules are bound to sites having strong interactions i.e. sites which are better suited to bind to the target molecules, among all the sites of the MIP which sites are able to interact with the target molecules.

After the sites of the MIP which sites are better suited to bind to the target molecules are bound to the target molecules to form a complex, the blocking step S2 and the second refining step S8 are carried out in this order, as illustrated in FIG. 5. Note that the blocking step S2 and the second refining step S8 are identical with the blocking step S2 and the refining step S3 of the First Embodiment, respectively, and explanations thereof are omitted here.

Thus, according to the producing method of the present embodiment, after target molecules are removed from an MIP complex having been bound to the target molecules, an obtained MIP and target molecules are caused to interact with each other to form a complex. In forming the complex, the MIP is interacted with target molecules the number of which is smaller than a total number of sites that exist in the MIP obtained by the first refining step S6 and are able to interact with the target molecules. In the complex obtained in this manner, the target molecules are bound to the sites of the MIP which sites are better suited to bind to the target molecules. In the present embodiment, free functional groups in such a complex are blocked. Thus, a finally obtained MIP according to the present invention realizes a higher selectivity than an MIP obtained by the producing method of the First Embodiment.

Note that the producing method of the present invention may include the blocking step between the polymerization step S1 and the first refining step S6 in FIG. 5.

Further, in the present invention, after (i) a target molecule recognition polymer that is capable of forming a complex together with a target molecule and (ii) target molecules in a predetermined quantity are mixed to form a complex, a deactivation step may be included in which functional groups which are not bound to the target molecules in the complex are deactivated.

<Use of MIP>

The MIP obtained by the producing method of the present invention has a high selectivity. Thus, even when high molecular weight molecules like biomolecules are used as the target molecules, the MIP obtained by the producing method of the present invention is able to bind to the target molecules with high accuracy.

In recent years, hazardous chemical substances have been released into environments including air, river water, and underground water. Examples of the hazardous chemical substances include environmental hormones (endocrine disrupting chemicals), their derivatives and similar compounds, and many agricultural chemicals. At the moment, there is a lot of uncertainty about the effects of such chemical substances on the human body and the ecosystem. However, since concerns about chronic toxicity caused by food chain have been rising, development of techniques for efficiently removing such chemical substances has been desired eagerly.

The MIP of the present invention is able to bind to target molecules with accuracy even when the target molecules are high molecular weight molecules like environmental hormones. That is, if the MIP of the present invention is produced by using particular environmental hormones as target molecules, the MIP thus produced can be used as a remover (adsorbent) of such target molecules.

Further, separation and extraction of a trace quantity of biological samples or the like become possible.

In view of this, the MIP of the present invention can be used for various purposes such as a biosensor, a chemical sensor, transportation, and condensation. Thus, it is considered that the MIP of the present invention can be used for various chemical industries such as manufacture of pharmaceutical products and manufacture of industrial chemicals, and moreover health care industry.

The present invention is not limited to the description of the embodiments above, but may be altered by a skilled person within the scope of the claims. An embodiment based on a proper combination of technical means disclosed in different embodiments is encompassed in the technical scope of the present invention.

Note that it can also be said that a method for producing a target molecule recognition polymer according to the present invention has the following arrangement:

That is, a method for producing a target molecule recognition polymer according to the present invention is a method for producing a molecular recognition polymer, including, after polymerization of a mixture of target molecules, functional monomers, and a crosslinking agent, a step of deactivating free functional groups in a complex of (a) target molecules and (b) a molecular recognition polymer for the target molecules, which complex has been obtained by the polymerization. Further, it can also be said that another method for producing a target molecule recognition polymer according to the present invention is a method for producing a molecular recognition polymer, including, after obtaining the molecular recognition polymer by refining, a step of mixing target molecules in extra-low concentration and the molecular recognition polymer for the target molecules so as to form a complex, and a step of deactivating free functional groups in the complex of (a) the target molecules and (b) the molecular recognition polymer for the target molecules.

Further, the above method can include, after polymerization of a mixture of target molecules, functional monomers, and a crosslinking agent, a step of cleaning, with a buffer solution, a complex of (a) target molecules and (b) a molecular recognition polymer for the target molecules, which complex has been obtained by the polymerization.

Further, the above method can include, after polymerization of a mixture of target molecules, functional monomers, and a crosslinking agent, a step of carrying out incomplete refining a complex of (a) target molecules and (b) a molecular recognition polymer for the target molecules, which complex has been obtained by the polymerization.

Further, regarding these methods, a deactivation material can be low molecular weight molecules having primary amine such as ethanolamine or tris(hydroxymethyl)aminomethane.

Still further, the deactivation material can be acetic anhydride.

Apart from the deactivation material, a polar low molecular weight molecule deactivation material such as polyvinyl alcohol or polyethyleneglycol can be used.

Further, it can be also said that target molecule recognition polymers according to the present invention have the following structure:

That is, a target molecule recognition polymer according to the present invention is such that in a process of preparation of a molecular recognition polymer for a target molecule, redundant functional groups in a molecular recognition polymer are deactivated.

Further, the target molecules may be high molecular weight molecules each having a polarity like biomolecule's polarity.

The following will describe details of the present invention on the basis of Example and Comparative Example. However, the present invention is not limited to the descriptions.

### EXAMPLE

Selection efficiency of (a) the MIP produced by the producing method of he present invention and (b) a comparative MIP produced by the producing method which does not include blocking step (deactivation step) was verified by experiment. To begin with, four types of MIPs (MIP-1 to MIP-4) produced by the producing method of the present invention and comparative MIPs are explained.

[MIP-1]

As shown in Table 1, glucose oxidase (hereinafter referred to as GOD) (commercially available) as target molecules was put on a chartula by measurement and then placed via the chartula into a 25 ml-vial. Thereafter, 10 ml of 12 mM phosphate buffer (pH 5.6) was added to GOD by using a micro pipette. Then, as shown in Table 1, 65 μl of acrylic acid as functional monomers (commercially available) was measured by a micro pipette and then placed into the vial. At this moment, a final concentration of GOD was approximately 1×10⁻⁶ M. The mixture was stirred well for 5 minutes to obtain target molecule bound monomers.

Next, as shown in Table 1, (i) acrylamide as supplementary monomers and (ii) N,N′-methylenebisacrylamide and N,N′-(1,2-dihydroxyethylene)-bisacrylamide as crosslinking agents (both commercially available) were put on a chartula by measurement and then placed via the chartula into the vial. Nitrogen gas was fed into the vial at 0.1 MPa, to carry out nitrogen substitution for 5 minutes.

Further, as shown in Table 1, 40% ammonium persulfate solution as a polymerization initiator and tetramethylethylenediamine (TEMED) as a polymerization accelerator were measured and placed into the vial. The resultant mixture solution was stood still at room temperature for 3 hours to promote polymerization reaction in the mixture solution. As a result, an MIP complex was obtained (polymerization step S1: FIG. 1)

After the polymerization, a solvent was removed by suction filtration from the obtained MIP complex. Thereafter, to the resultant MIP complex, 10 ml of 1M ethanolamine solution was added to carry out blocking process (blocking step S2: FIG. 1).

One hour after the ethanolamine solution was added, ethanolamine was removed with a buffer shown in Table 1. Thereafter, GOD bound to the MIP complex was removed from the residue by using 120 mM phosphate buffer (pH 5.6) as a refining solution to obtain an MIP (refining step S3: FIG. 1).

TABLE 1
MIP-1

| | | |
|---|---|---|
| Target molecule: glucose oxidase | 16 | mg |
| Functional monomer: acrylic acid | 65 | μl |
| Supplementary monomer: acrylamide | 1000 | mg |
| Crosslinking agents: | | |
| N,N′-methylenebisacrylamide | 130 | mg |
| N,N′-(1,2-dihydroxyethylene)-bisacrylamide | 120 | mg |
| Polymerization accelerator: TEMED | 30 | μl |
| Polymerization initiator: 40% ammonium persulfate | 250 | μl |
| Buffer: 12 mM phosphate buffer (pH 5.6) | 10 | ml |
| Refining solution: 120 mM phosphate buffer (pH 5.6) | 50 | ml |

[MIP-2]

The polymerization process and the previous processes were performed as in the case of MIP-1 to prepare an MIP complex (polymerization step S1: FIG. 3).

After the MIP complex was formed, a solvent was removed from the MIP complex by suction filtration. Subsequently, as shown in Table 2, the resultant MIP complex was subjected to suction filtrations with 10 ml of cleaning solution five times to wash away (a) acrylic acid (functional monomers) and GOD both of which had remained in a polymerization reaction solution without making up the MIP complex and (b) GOD which had been physically adsorbed with the MIP complex (cleaning step S4: FIG. 3).

Thereafter, as in the case of MIP-1, 1M ethanolamine solution was added to the MIP complex to perform blocking reaction for 1 hour (blocking step S2: FIG. 3).

Subsequently, as in the case of MIP-1, ethanolamine was removed from the MIP complex by using a buffer shown in Table 2. Thereafter, GOD was removed from the MIP complex by using a refining solution to obtain an MIP (refining step S3: FIG. 3).

TABLE 2
MIP-2

| | | |
|---|---|---|
| Target molecule: glucose oxidase | 16 | mg |
| Functional monomer: acrylic acid | 65 | μl |
| Supplementary monomer: acrylamide | 1000 | mg |
| Crosslinking agents: | | |
| N,N′-methylenebisacrylamide | 130 | mg |
| N,N′-(1,2-dihydroxyethylene)-bisacrylamide | 120 | mg |
| Polymerization accelerator: TEMED | 30 | μl |
| Polymerization initiator: 40% ammonium persulfate | 250 | μl |
| Cleaning solution: 12 mM phosphate buffer (pH 5.6) | 10 | ml |
| | each | time |
| Buffer: 12 mM phosphate buffer (pH 5.6) | 10 | ml |
| Refining solution: 120 mM phosphate buffer (pH 5.6) | 50 | ml |

[MIP-3]

The polymerization process and the previous processes were performed as in the case of MIP-1 to prepare an MIP complex (polymerization step S1: FIG. 4).

After the MIP complex was formed, a solvent was removed from the MIP complex by suction filtration. Subsequently, as shown in Table 3, the MIP complex was subjected to suction filtrations with 10 ml of pretreatment solution twice to remove GOD which had been bound to the MIP complex by weak interaction from the MIP complex, and remove (a) acrylic acid (functional monomers) and GOD both of which had remained in a polymerization reaction solution without making up the MIP complex and (b) GOD which had been physically adsorbed with the MIP complex (pretreatment step S5: FIG. 4).

Thereafter, the MIP complex was cleaned with a buffer (10 ml each time) shown in Table 3 six times. Then, as in the case of MIP-1, 1M ethanolamine solution was added to the MIP complex to perform blocking reaction for 1 hour (blocking step S2: FIG. 4).

Subsequently, as in the case of MIP-1, ethanolamine was removed from the MIP complex by using a buffer shown in Table 3. Thereafter, GOD was removed from the MIP complex by using a refining solution to obtain an MIP (refining step S3: FIG. 4).

TABLE 3
MIP-3

| | | |
|---|---|---|
| Target molecule: glucose oxidase | 16 | mg |
| Functional monomer: acrylic acid | 65 | μl |
| Supplementary monomer: acrylamide | 1000 | mg |
| Crosslinking agents: | | |
| N,N′-methylenebisacrylamide | 130 | mg |
| N,N′-(1,2-dihydroxyethylene)-bisacrylamide | 120 | mg |
| Polymerization accelerator: TEMED | 30 | μl |
| Polymerization initiator: 40% ammonium persulfate | 250 | μl |
| Pretreatment solution: acetate buffer (pH 2.0) | 10 | ml |
| | each | time |
| Buffer: 12 mM phosphate buffer (pH 5.6) | 10 | ml |
| Refining solution: 120 mM phosphate buffer (pH 5.6) | 50 | ml |

[MIP-4]

The polymerization process and the previous processes were performed as in the case of MIP-1 to prepare an MIP complex (polymerization step S1: FIG. 5).

After the MIP complex was formed, a solvent was removed by suction filtration. Then, GOD was removed from the MIP complex by using a refining solution shown in Table 4 to obtain an MIP (first refining step S6: FIG. 5).

Next, the obtained MIP having a final concentration of 2×10⁻⁷ M was mixed with GOD having a final concentration of 2×10⁻⁷ M (total volume: 10 ml) to obtain GOD-polymer complex (target molecule-polymer complex) (complex forming step S7: FIG. 5).

Thereafter, as in the case of MIP-1, 1M ethanolamine solution was added to the MIP complex to perform blocking reaction for 1 hour (blocking step S2: FIG. 5).

Subsequently, as in the case of MIP-1, ethanolamine was removed from the MIP complex by using a buffer shown in Table 4. Thereafter, GOD was removed from the MIP complex by using a refining solution to obtain an MIP (refining step S3: FIG. 5).

TABLE 4
MIP-4

| Target molecule: glucose oxidase | 16 | mg |
|---|---|---|
| Functional monomer: acrylic acid | 65 | μl |
| Supplementary monomer: acrylamide | 1000 | mg |
| Crosslinking agents: | | |
| N,N′-methylenebisacrylamide | 130 | mg |
| N,N′-(1,2-dihydroxyethylene)-bisacrylamide | 120 | mg |
| Polymerization accelerator: TEMED | 30 | μl |
| Polymerization initiator: 40% ammonium persulfate | 250 | μl |
| Glucose oxidase for complex formation | Final | |
| | concentration of | |
| | 2 × 10⁻⁷ M | |
| Buffer: 12 mM phosphate buffer (pH 5.6) | 10 | ml |
| Refining solution: 120 mM phosphate buffer (pH 5.6) | 50 | ml |

[Comparative MIP]

The polymerization process and the previous processes were performed as in the case of MIP-1 to prepare an MIP complex. Thereafter, a solvent was removed from the MIP complex by suction filtration.

Subsequently, GOD was removed from the MIP complex by using a refining solution to obtain an MIP.

TABLE 5
Comparative MIP

| | | |
|---|---|---|
| Target molecule: glucose oxidase | 16 | mg |
| Functional monomer: acrylic acid | 65 | μl |
| Supplementary monomer: acrylamide | 1000 | mg |
| Crosslinking agents: | | |
| N,N′-methylenebisacrylamide | 130 | mg |
| N,N′-(1,2-dihydroxyethylene)-bisacrylamide | 120 | mg |
| Polymerization accelerator: TEMED | 30 | μl |
| Polymerization initiator: 40% ammonium persulfate | 250 | μl |
| Buffer: 12 mM phosphate buffer (pH 5.6) | 10 | ml |
| Refining solution: 120 mM phosphate buffer (pH 5.6) | 50 | ml |

[Experiment of Binding]

By using the MIP-1 to MIP-4 prepared as above and the comparative MIP and glucose oxidase as target molecules, the quantities of the MIPs which were bound to glucose 6-phosphate dehydrogenase (G6PD) as non-target molecules were measured.

For each of the prepared MIPs, two 10 ml-vials were prepared. One had a GOD solution of 5 ml (concentration of 30 μM) in which GOD is dissolved in 12 mM phosphate buffer (pH 4.2), and the other had a G6PD solution of 5 ml in which G6PD was dissolved in 12 mM phosphate buffer (pH 4.2). As to each of the MIPs, 10 mg of the MIP was measured and added into each of the vials, and the resultant solutions were stood still at 25° C. for 30 minutes so that binding reaction occurred.

Thereafter, the resultant solutions were centrifuged at 1000 rpm for five minutes to precipitate the MIP and obtain a supernatant. From enzyme activity of the supernatant, the amount of GOD bound to the MIP and the amount of G6PD bound to the MIP were measured.

The results of the measurement are as shown in Table 6. Table 6 shows the amount of GOD bound to 1 g of the MIP, the amount of G6PD bound to Ig of the MIP, and selection efficiency of the MIP, for each of the MIPs.

TABLE 6

| | Unit: μmol/g | | | | |
|---|---|---|---|---|---|
| | | | | | Comparative |
| | MIP-1 | MIP-2 | MIP-3 | MIP-4 | MIP |
| Amount of | 10 | 8 | 6 | 6 | 12 |
| bound GOD (i) | | | | | |
| Amount of | 4 | 3 | 2 | 2 | 8 |
| bound G6PD | | | | | |
| Selection | 2.5 | 2.6 | 3 | 3 | 1.5 |
| efficiency (i)/(ii) | | | | | |

The results shown in Table 6 indicated that selection efficiencies of the MIP-1 to MIP-4 are higher than that of the comparative MIP. That is, it was confirmed that the MIPs produced by the producing method of the present invention realized enhancement of selectivity with respect to a target molecule, in comparison with the MIP produced by the conventional method.

Further, it was confirmed that selection efficiencies of the MIP-2 to MIP-4 were higher than that of the MIP-1. This indicates that a producing method in which the blocking step is carried out after the cleaning step (S4: FIG. 3), the pretreatment step (S5: FIG. 4), the complex forming step (S7: FIG. 5) further enhanced selectivity of the MIP.

The embodiments and concrete examples of implementation discussed in the foregoing detailed explanation serve solely to illustrate the technical details of the present invention, which should not be narrowly interpreted within the limits of such embodiments and concrete examples, but rather may be applied in many variations within the spirit of the present invention, provided such variations do not exceed the scope of the patent claims set forth below.

What is claimed is:

1. A method for producing a target molecule recognition polymer, the method comprising:
a polymerization step of polymerizing a mixture of one or more target molecules and functional monomers having functional groups, which are able to interact with the target molecules, so as to form a target molecule recognition polymer complex to which target molecules are bound; and
a refining step of refining the target molecule recognition polymer complex by having one or more target molecules removed from the target molecule recognition polymer complex, so as to obtain a target molecule recognition polymer,
the method further comprising:
a deactivation step of deactivating a functional group which is contained in the target molecule recognition polymer complex but is not bound to the target molecule.

2. The method according to claim 1, further comprising:
a cleaning step of removing (a) a target molecule which is physically adsorbed with the target molecule recognition polymer complex and/or (b) a target molecule and a functional monomer both of which have not made up the target molecule recognition polymer complex.

3. The method according to claim 1, wherein
in the refining step used is a dissociation solution which dissociates the target molecules from the target molecule recognition polymer complex at a predetermined dissociation strength, and
the method further comprises: a pretreatment step of, by using a solution having a dissociation strength lower than the predetermined dissociation strength, removing (a) a target molecule which is physically adsorbed with the target molecule recognition polymer complex and/or (b) part of target molecules which is bound to the target molecule recognition polymer complex by interaction with the functional group.

4. The method according to claim 1, wherein
primary amine having a molecular weight of not more than several thousands is used in the deactivation step.

5. The method according to claim 4, wherein
the primary amine is ethanolamine or tris(hydroxymethyl)aminomethane.

6. The method according to claim 4, wherein acetic anhydride is used in the deactivation step.

7. The method according to claim 1, wherein
a water-soluble polymer is used in the deactivation step.

8. The method according to claim 7, wherein
the water-soluble polymer is polyvinyl alcohol or polyethyleneglycol in the deactivation step.

9. The method according to claim 1, wherein
the target molecules are high molecular weight molecules each having a molecular weight of not less than ten thousands.

10. The method according to claim 1, wherein
the target molecules are biomolecules.

11. The method according to claim 1, wherein
a crosslinking agent is further mixed in the polymerization step.

12. A target molecule recognition polymer produced by a producing method comprising: a polymerization step of polymerizing a mixture of one or more target molecules and functional monomers having functional groups, which are able to interact with the target molecules, so as to form a target molecule recognition polymer complex to which target molecules are bound; a refining step of refining the target molecule recognition polymer complex by having one or more target molecules removed from the target molecule recognition polymer complex, so as to obtain a target molecule recognition polymer; and a deactivation step of deactivating a functional group which is contained in the target molecule recognition polymer complex but is not bound to the target molecule.

13. A method for producing target molecule recognition polymers, the method comprising:
a polymerization step of polymerizing a mixture of one or more target molecules and functional monomers having functional groups, which are able to interact with the target molecules, so as to form a target molecule recognition polymer complex to which target molecules are bound;
a refining step of refining the target molecule recognition polymer complex by having one or more target molecules removed from the target molecule recognition polymer complex, so as to obtain a target molecule recognition polymer;
a complex forming step of mixing the target molecule recognition polymer that has been obtained in the refining step with target molecules in predetermined quantity, so as to form a complex of the target molecule recognition polymer with a target molecule; and
a deactivation step of deactivating a functional group which is contained in the target molecule recognition polymer complex that has been obtained in the complex forming step, but is not bound to the target molecule.

14. The method according to claim 13, further comprising:
a cleaning step of removing (a) a target molecule which is physically adsorbed with the target molecule recognition polymer complex and/or (b) a target molecule and a functional monomer both of which have not made up the target molecule recognition polymer complex.

15. The method according to claim 13, wherein
in the refining step used is a dissociation solution which dissociates the target molecules from the target molecule recognition polymer complex at a predetermined dissociation strength, and
the method further comprises: a pretreatment step of, by using a solution having a dissociation strength lower than the predetermined dissociation strength, removing (a) a target molecule which is physically adsorbed with the target molecule recognition polymer complex and/or (b) part of target molecules which is bound to the target molecule recognition polymer complex by interaction with the functional group.

16. The method according to claim 13, wherein
primary amine having a molecular weight of not more than several thousands is used in the deactivation step.

17. The method according to claim 16, wherein
the primary amine is ethanolamine or tris(hydroxymethyl)aminomethane.

18. The method according to claim 13, wherein
acetic anhydride is used in the deactivation step.

19. The method according to claim 13, wherein
a water-soluble polymer is used in the deactivation step.

20. The method according to claim 19, wherein
the water-soluble polymer is polyvinyl alcohol or polyethyleneglycol in the deactivation step.

21. The method according to claim 13, wherein
the target molecules are high molecular weight molecules each having a molecular weight of not less than ten thousands.

22. The method according to claim 13, wherein
the target molecules are biomolecules.

23. The method according to claim 13, wherein
a crosslinking agent is further mixed in the polymerization step.

24. A target molecule recognition polymer produced by a producing method comprising: a polymerization step of polymerizing a mixture of one or more target molecules and functional monomers having functional groups, which are able to interact with the target molecules, so as to form a target molecule recognition polymer complex to which target molecules are bound; a refining step of refining the target molecule recognition polymer complex by having one or more target molecules removed from the target molecule recognition polymer complex, so as to obtain a target molecule recognition polymer; a complex forming step of mixing the target molecule recognition polymer that has been obtained in the refining step with target molecules in predetermined quantity, so as to form a complex of the target molecule recognition polymer with a target molecule; and a deactivation step of deactivating a functional group which is contained in the target molecule recognition polymer complex that has been obtained in the complex forming step, but is not bound to the target molecule.

25. A sensor for detecting a target molecule, the sensor comprising target molecule recognition polymer produced by a producing method comprising: a polymerization step of polymerizing a mixture of one or more target molecules and functional monomers having functional groups, which are able to interact with the target molecules, so as to form a target molecule recognition polymer complex to which target molecules are bound; a refining step of refining the target molecule recognition polymer complex by having one or more target molecules removed from the target molecule recognition polymer complex, so as to obtain a target molecule recognition polymer; and a deactivation step of deactivating a functional group which is contained in the target molecule recognition polymer complex but is not bound to the target molecule.

26. A sensor for detecting target molecules, the sensor comprising target molecule recognition polymers produced by a producing method comprising: a polymerization step of polymerizing a mixture of one or more target molecules and functional monomers having functional groups, which are able to interact with the target molecules, so as to form a target molecule recognition polymer complex to which target molecules are bound; a refining step of refining the target molecule recognition polymer complex by having one or more target molecules removed from the target molecule recognition polymer complex, so as to obtain a target molecule recognition polymer; a complex forming step of mixing the target molecule recognition polymer that has been obtained in the refining step with target molecules in predetermined quantity, so as to form a complex of the target molecule recognition polymer with a target molecule; and a deactivation step of deactivating a functional group which is contained in the target molecule recognition polymer complex that has been obtained in the complex forming step, but is not bound to the target molecule.

27. A Target molecule recognition polymer obtained by polymerizing functional monomers having functional groups, which are able to interact with target molecules, wherein part of the functional groups is deactivated.

28. A sensor for detecting a target molecule, the sensor comprising a target molecule recognition polymer obtained by polymerizing functional monomers having functional groups, which are able to interact with target molecules, wherein part of the functional groups is deactivated.
